(19) United States
(12) Patent Application Publication
(10) Pub. No.: US 2003/0039682 A1
(43) Pub. Date: Feb. 27, 2003

(54) Article such as a makeup removal pad comprising an external surface for applying aqueous products on the skin

(76) Inventor: Philippe Gregoire, Les Andelys (FR)

Correspondence Address: Breiner & Breiner, 115 North Henry Street, PO Box 19290, Alexandria, VA, 22320-0290, US

(21) Appl. No.: 10/148,918

(22) PCT Filed: Dec. 5, 2000
(86) PCT No.: PCT/FR00/03394

(51) Int. Cl. A61K 9/70; B32B 27/12; B32B 27/04

(52) U.S. Cl. 424/443; 442/118

(57) ABSTRACT

The invention relates to a product such as a makeup-removal pad based on hydrophilic cotton fibers and including an external side used to apply aqueous substances to the skin. In the invention, the external side is treated with an agent delaying absorption of the aqueous substances. The product of the invention is used to apply personal care substances, such as cosmetics, to the skin.

The present invention relates to a cotton product for skin care, such as a makeup-removal pad, comprising an external surface with which to apply aqueous substances to the skin.

This cotton product is a product or pad cut into round formats of oval, square or any other shape commonly called “makeup removal disks”.

In the text below, the expression “pad based on hydrophilic cotton fibers” shall include any product essentially comprising hydrophilic and absorbent cotton fibers in a proportion from 70 to 100% of cotton fibers and from 0 to 30% of artificial fibers such as viscose fibers or synthetic fibers such as polyolefin fibers or binary fibers (for example polyester/polyester, polyester/polypropylene, polypropylene/polyethylene).

The specific surface weight of the cotton product ranges from 150 to 400 g/m² and preferably from 180 to 300 g/m².

Skin care includes body care, face care and more specifically beauty care using cosmetics, namely removing makeup from and applying makeup to the face, furthermore baby care, for example changing the baby, etc.

On account of the hydrophilic nature of the cotton fibers, one major drawback of the pad or cotton products is their excessive absorption of the substances applied to their surface. In general, an aqueous substance enters the inside of the pad by capillarity and sometimes it even crosses the pad and moistens the hands.

The care substance is used wastefully and the pad, being impregnated with the substance, will deform.

Solutions to the problem of the care substance fully crossing the pad already have been advanced.

For example, one solution is described in U.S. Pat. No. 5,230,119 and consists in a grip-fitted pad compressing a rear layer in the form of a thin, impermeable plastic film or of an impermeable treated paper foil.

Another solution is disclosed in European Patent 0 441 667 and consists in using a makeup-removal pad comprising a central hydrophobic blocking layer and/or one which is impermeable relative to the substance to be removed, two absorbent layers sandwiching the former one and adhering to it. Preferably the absorbent material is carded or combed cotton. The blocking layer may consist of a stratum of one or several flexible plastic materials such as polyethylene, polypropylene or polyester.

However, as regards the above solutions, the impermeable or blocking layers were deposited rather than being made of the absorbent material or of the web constituting the pad. They are wholly impermeable and must not come into contact with the skin.

By controlling the kind of fibers used, it is possible furthermore to modify the properties of the cotton pad or also one of its external layers.

Illustratively, hydrophobic olefin fibers allowing carding may be mixed with hydrophilic cotton fibers. Such fibers are described in European Patent 0 687 318. But in such a case the cotton product's fiber composition changes.

Modifying neither composition nor structure of the product, U.S. Pat. No. 5,480,699 describes mechanical compression to prevent large amounts of liquid or cream from penetrating the pad. By more strongly compressing one of the external layers constituted of a carded web, and by joining the two carded webs to an uncompressed central layer, the final product exhibits one surface which is more compressed and wherein the fibers are tighter together, leaving fewer interstices to transmit creams or liquids to be applied to the skin. This solution, however, still allows some liquid or cream to reach the inside of the pad through the remaining interstices because the compression also improves capillarity. Absorption is not delayed in this kind of product.

The problem addressed by the present invention is to delay the absorption of aqueous substances at one of the cotton product's sides while irreversibly making the latter impermeable and without modifying the fiber composition of the product.

The “impermeabilization” of the invention is temporary and its purpose is to preclude substances from penetrating the cotton pads at the time of the substances' application on the pads and to delay absorption of substances, in this manner allowing substance application to the skin before there is substance penetration of the cotton pad.

The objective of the invention is to create a cotton product, such as a makeup-removal pad, substantially composed of hydrophilic cotton fibers, and in this manner solve the above stated problem of efficiently applying aqueous substances to the skin.

In one essential feature of the invention, one of the external sides of the cotton product comprises an agent delaying the absorption of aqueous substances.

In one advantageous feature of the invention, the aqueous substances' absorption delaying agent is in the form of an emollient- or a wax-based composition.

In another feature of the invention, the absorption delaying agent is a component attaching itself to the fibers, for example in the form of complex metal salts of stearic acid, perfluorinated derivatives, zirconium salts or silicones.

In another preferred embodiment of the invention, the agent is a natural, mineral, vegetal or animal wax.

In an especially preferred feature of the invention, the agent is a beeswax emulsion.

In another feature of the invention, the agent is an emulsion or a dispersion containing at least 30% active ingredients.

In another advantageous feature of the invention, the first side contains at least 0.3 g/m² of active ingredients.

Other features and advantages are elucidated in the description below.

In order to optimize applying a substance to the skin or the cleansing of the skin, removing makeup and/or applying it using cosmetics, the product or cotton pad of the invention includes an agent which delays absorption of aqueous substances at one of its external sides which hereafter is called the first external side.

The absorption delaying agent is a component based on emollients or waxes, or also a component attaching itself to the fibers.

When the components are applied in small quantities to conventionally hydrophilic and absorbent cotton products, they can be observed as being surprisingly effective in delaying the absorption of aqueous substances deposited on the surface of the cotton products.

Illustratively, the emollients are fatty amines, fatty alcohols, fatty esters, fatty polyethylenes or polyamides or their mixtures.

Illustratively, the components attaching themselves to the fibers are complex metal salts of stearic acid, perfluorinated derivatives, zirconium salts or silicones.

The wax-based components are wax and paraffin emulsions, also emulsions of wax only.

Preferably the aqueous substances absorption delaying agent is be a natural wax emulsion of mineral, vegetal or animal origin.

Examples of animal origin waxes are spermaceti and bee waxes.

Examples of vegetal origin waxes are candellila and carnauba waxes.

Examples of mineral origin waxes are ceresin and azocerite.

Beeswax emulsion is especially advantageous and appropriate to the pad's cosmetic use, being dermatologically tested already. This is a cationic, bleached beeswax emulsion containing beeswax, water, emulsifiers, glyceryl stearate and diethanolaminoether stearate.

Beeswax per se is composed of waxy esters of fatty acids such as myricyl palmitate, cerotic acid and other homologous waxy acids, and of small quantities of hydrocarbons, of cholesterol esters and of ceryl alcohols.

The absorption delaying agent is an emulsion or dispersion containing at least 30% active ingredients.

The first side of the product, or cotton pad, of the invention contains at least 1 g/m² of applied emulsion, that is at least 0.3 g/m² of deposited active ingredients.

The first side fitted at its surface with the agent thereby offers advantageous properties.

It delays the penetration of the aqueous substances applied to the pad's surface.

Penetration by the care substances, such as toiletry milk, makeup or makeup removal substances, represents a major drawback of conventional hydrophilic cotton pads. The care or cosmetic substances are wasted, they are used uneconomically, sometimes they cross the pad. Skin cleansing efficiency is less than optimal.

The treatment of the invention of the pad's first side creates a temporary water “impermeability” and thus allows averting the nearly instant absorption of aqueous substances by the hydrophilic cotton fibers as soon as such aqueous substances are deposited on the pad.

A simple test proving this property consists in depositing, at ambient temperature (about 20° C.), pads processed in the manner of the invention at the surface of a water-filled container, the treated side pointing outward and the absorbing side toward the water, and further depositing conventional pads consisting of 100% cotton fibers. The former pads remain at least 5 minutes at the water surface whereas the latter pads are nearly immediately water-logged and immerse rapidly, in general after 3-5 seconds.

The advantage offered by the invention is that they retain substances longer at the surface and make full use of the quantity of substance deposited on the pad as regards skin care, without loss of substance and without deforming the pad.

A test regarding skin care, makeup removal etc. was carried out at applicant's premises on 25 persons ordinarily and exclusively using conventional hydrophilic cotton pads B.

The B pads are 100% cotton fibers and made from cotton webs manufactured according to European Patent Application 0 735 175 and are marketed as DEMAK'UP®.

Pads A of the invention having the first side treated with an absorption delaying agent, more specifically with a beeswax emulsion, were tested relative to the conventional B pads.

The following test results were ascertained:

As regards the A pads of the invention, of which the first side had been treated, almost all persons, namely 92%, noted the pad's ability to delay the absorption of the care substances deposited at the pads' surface.

Regarding the pads of the invention with their treated first surfaces, the number of persons noticing cleansing improvement is 92%.

With respect to makeup removal, 85% of the persons noted improved cleansing efficacy of the pads of the invention that were treated on the first surface.

Similar advantages were noted when using makeup substances such as lotions, creams, makeup foundations, rouge, in applying on and spreading the substance over the skin.

The persons also advantageously used the product of the invention to apply perfumes such as toilet water. Toilet water is not immediately absorbed by the pad of the invention compared to the case of applying toilet water by means of cotton products of the prior art.

The product, or cotton pad, of the invention consists of one or more layers of cotton fibers manufactured by lapping, carding or in a pneumatic manner.

These layers may exhibit identical or different fiber qualities. They may be manufactured directly from hydrophilic and bleached cotton. They also may be made from crude, unbleached cotton subsequently treated chemically. Next, these layers are superposed and combined by any known means, such as glueing or by mechanical means such a calendering or needling. The combining means also may be hydraulic.

Good combination may be attained by impregnation, spraying, or by pouring a solution. The impregnation is combined with expression compacting the layer and eliminating part of the liquid contained in the moist layer, for example by calendering or moving past a vacuum slot.

The hydrolinking technique allows both combining the two layers and linking of the layer's surfaces.

Hydrolinking treatment may be applied in the case of a crude layer that is chemically treated immediately after the layer's impregnation stage in the manner described in European Patent Application 0 735 175.

The hydrolinking stage also may take place in the final rinsing procedure described in applicant's European Patent 0 805 888 wherein the manufacture of the cotton sheet is in a continuous manufacturing line.

When manufacturing the product of the invention, one of the external sides (first external side) of the cotton product is treated in a manner to delay absorption of aqueous substances (care substances . . . ) when the pads are used for cosmetic purposes.

Following the impregnation stages, and before or after drying, the first side is treated by applying to it an agent delaying absorption of aqueous substances as described above. In a preferred embodiment of the invention, wax emulsion is applied at the rate of at least 1 g/m², that is at least 0.3 g/m² of active ingredient (waxes), at the product surface.

This surface treatment is carried out using any conventional means such as nozzle spraying, coating using a cylinder, rotogravure printing, . . .

In order to better tell the difference between the treated and untreated surfaces, the product of the invention may employ visual differentiation of the two sides, for example one side being fitted with a pattern and the other not, or two sides being imprinted in a different manner with different patterns, or again coloring one of the sides, etc.

The products so made and fitted with sides that differ from each other on account of the surface treatment of one of the external sides shall then be cut to format and packed into flexible wraps or pouches.

1. A product such as a makeup-removal pad based on hydrophilic cotton fibers, comprising an external side with which to apply aqueous substances onto the skin, characterized in that the said external side is fitted with an agent delaying absorption of aqueous substances.

2. Product as claimed in claim 1, characterized in that the agent delaying absorption of aqueous substances is a composition based on emollients or waxes.

3. Product as claimed in claim 2, characterized in that the emollients include fatty amines, fatty alcohols, fatty esters, fatty polyethylenes or polyamides, or their mixtures.

4. Product as claimed in claim 2, characterized in that the agent delaying absorption of aqueous substances is a natural wax emulsion of mineral, vegetal or animal origin.

5. Product as claimed in claim 4, characterized in that the animal origin wax is spermaceti wax or preferably beeswax.

6. Product as claimed in claim 4, characterized in that the vegetal origin wax is candellila or carnauba wax.

7. Product as claimed in claim 4, characterized in that the mineral origin wax is ceresin or azocerite.

8. Product as claimed in claim 1, characterized in that the agent delaying absorption of aqueous substances is a component attaching itself to the fibers, for instance in the form of complex metal salts of stearic acid, perfluorinated derivatives, zirconium salts or silicones.

9. Product as claimed in one of the above claims, characterized in that said agent is an emulsion or dispersion containing at least 30% of active ingredients.

10. Product as claimed in claim 9, characterized in that the side designed to apply aqueous substances to the skin contains at least 0.3 g/m² of active ingredients.

11. Product as claimed in one of the above claims, characterized in that the aqueous substances illustratively are cosmetic substances in particular including makeup substances, makeup-removal substances, toiletry milks.
